(19) United States
(12) Patent Application Publication
(10) Pub. No.: US 2022/0151592 A1
(43) Pub. Date: May 19, 2022

(54) ULTRASONIC DIAGNOSTIC APPARATUS AND METHOD

(71) Applicant: Canon Medical Systems Corporation, Otawara-shi (JP)

(72) Inventors: Akio GODA, Otawara (JP); Akihiro KAKEE, Nasushiobara (JP)

(73) Assignee: Canon Medical Systems Corporation, Otawara-shi (JP)

(21) Appl. No.: 17/455,045

(22) Filed: Nov. 16, 2021

(30) Foreign Application Priority Data
Nov. 18, 2020 (JP) 2020-191960

(51) Int. Cl. A61B 8/08; A61B 8/00

(52) U.S. Cl. CPC A61B 8/481; A61B 8/54; A61B 8/488; A61B 8/5207

(57) ABSTRACT

According to one embodiment, an ultrasonic diagnostic apparatus includes processing circuitry. The processing circuitry determines whether or not to increase the number of beams to be compounded based on the information on the examination mode and increases the number of beams to be compounded when determining that the number of beams to be compounded is to be increased.

### CROSS-REFERENCE TO RELATED APPLICATIONS

This application is based upon and claims the benefit of priority from Japanese Patent Application No. 2020-191960, filed Nov. 18, 2020, the entire contents of which are incorporated herein by reference.

### FIELD

Embodiments described herein relate generally to an ultrasonic diagnostic apparatus and a method.

### BACKGROUND

Conventionally, in an ultrasonic diagnostic apparatus, a contrast echo method called contrast harmonic imaging (CHI) is performed. In the contrast echo method, for example, in an examination of the liver, heart, etc., a contrast medium is injected via a vein to perform imaging. Most of the contrast media used in the contrast echo method use microbubbles as a reflection source. By the contrast echo method, for example, a blood vessel in a subject can be clearly visualized, or the flow of the contrast medium in the blood vessel can be visualized.

Further, in an ultrasonic diagnostic apparatus, a technique called transmit aperture synthesis is known as a method of simultaneous parallel reception. Transmit aperture synthesis is a method of acquiring a plurality of received echo signals focused on the same observation point between transmission beams having different transmission focusing points and performing additive synthesis. By using the transmit aperture synthesis, in addition to improving S/N, it is possible to form a uniform transmission beam width in a depth direction with high accuracy, thereby generating an ultrasonic image with excellent spatial resolution and contrast resolution.

Thus, in the ultrasonic diagnostic apparatus, by performing the transmit aperture synthesis during the execution of the contrast echo method, it is considered that generation of a contrast image with excellent spatial resolution and contrast resolution can be expected and examination accuracy in a contrast examination can be improved. However, a control method for simultaneously performing the contrast echo method and the transmit aperture synthesis in the ultrasonic diagnostic apparatus is not known.

### BRIEF DESCRIPTION OF THE DRAWINGS

FIG. 1 is a block diagram showing a configuration example of an ultrasonic diagnostic apparatus according to a first embodiment.

FIG. 2 is a flowchart for explaining operations of a processing circuitry executing transmit aperture synthesis control processing in the first embodiment.

FIG. 3 is a diagram for explaining a flow of an examination in a contrast examination mode in the first embodiment.

FIG. 4 is a flowchart illustrating a beams to compound number determination process of the flowchart of FIG. 2.

FIG. 5 is a diagram showing a first display example of contrast image data in the first embodiment.

FIG. 6 is a diagram showing a second display example of contrast image data in the first embodiment.

FIG. 7 is a diagram for explaining a timer reset process in the first embodiment.

FIG. 8 is a diagram for explaining operations of transmit control through transmit aperture synthesis.

FIG. 9 is a diagram for explaining operations of receive control through transmit aperture synthesis.

### DETAILED DESCRIPTION

In general, according to one embodiment, an ultrasonic diagnostic apparatus includes processing circuitry. The processing circuitry determines whether or not to increase the number of beams to be compounded based on the information on the examination mode and increases the number of beams to be compounded when determining that the number of beams to be compounded is to be increased.

Hereinafter, embodiments of an ultrasonic diagnostic apparatus will be described in detail with reference to the drawings.

### First Embodiment

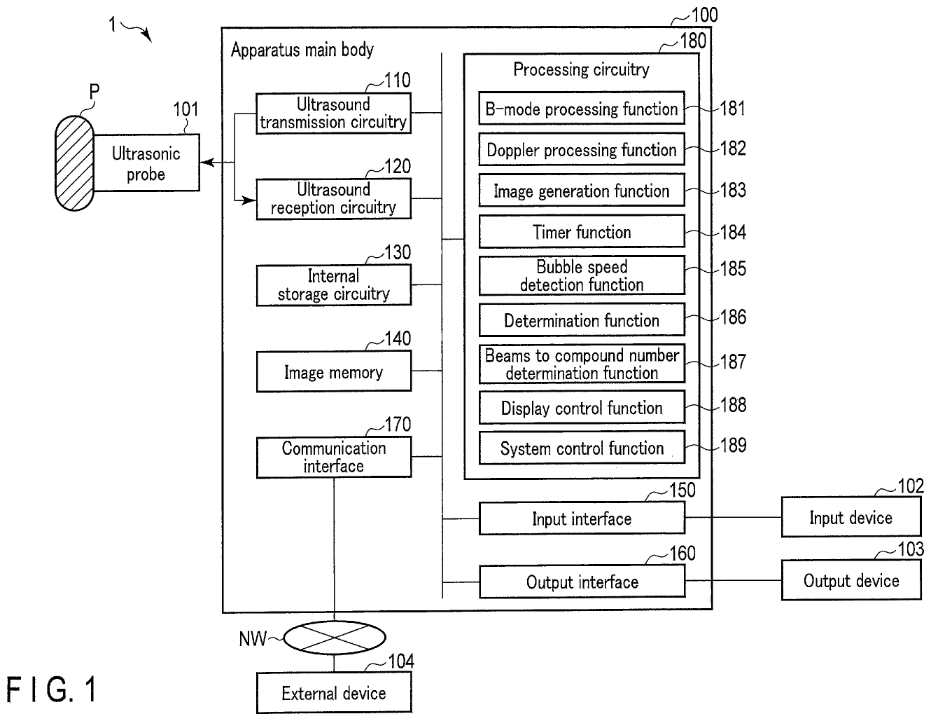

FIG. 1 is a diagram showing a configuration example of an ultrasonic diagnostic apparatus according to a first embodiment. An ultrasonic diagnostic apparatus 1 of FIG. 1 includes an apparatus main body 100 and an ultrasonic probe 101. The apparatus main body 100 is connected to an input device 102 and an output device 103. In addition, the apparatus main body 100 is connected to an external device 104 via a network NW. The external device 104 is, for example, a server equipped with picture archiving and communication systems (PACS) and a workstation capable of executing post processing.

The ultrasonic probe 101 executes, for example, ultrasonic scanning on a scan area in a living body P, which is a subject, under control of the apparatus main body 100. The ultrasonic probe 101 includes, for example, an acoustic lens, one or more matching layers, a plurality of vibrators (piezoelectric elements), a backing material, etc. The acoustic lens is made of, for example, silicone rubber, and converges ultrasonic beams. The one or more matching layers perform impedance matching between the plurality of vibrators and the living body. The backing material prevents propagation of ultrasonic waves backward in a radial direction from the plurality of vibrators. The ultrasonic probe 101 is, for example, a one-dimensional array linear probe in which a plurality of vibrators are arranged along a predetermined direction. The ultrasonic probe 101 is detachably connected to the apparatus main body 100. The ultrasonic probe 101 may be provided with a button which is to be pressed at the time of an offset process, an operation of freezing an ultrasonic image (freeze operation), etc.

The plurality of vibrators generate ultrasonic waves based on drive signals supplied from an ultrasound transmission circuitry 110 to be described later included in the apparatus main body 100. Thereby, ultrasonic waves are transmitted from the ultrasonic probe 101 to the living body P. When ultrasonic waves are transmitted from the ultrasonic probe 101 to the living body P, the transmitted ultrasonic waves are sequentially reflected by a discontinuous surface of acoustic impedance in a living tissue of the living body P, and received by the plurality of piezoelectric vibrators as reflected wave signals. An amplitude of the received reflected wave signal depends on a difference in acoustic impedance on the discontinuous surface by which the ultrasonic waves are reflected. When a transmitted ultrasonic pulse is reflected by a moving blood flow, a surface of a cardiac wall, etc., a frequency of the reflected wave signal is shifted, due to the Doppler effect, depending on a velocity component of a moving object in an ultrasonic transmission direction. The ultrasonic probe 101 receives the reflected wave signal from the living body P, and converts the reflected wave signal into an electric signal.

FIG. 1 illustrates a connection relationship between one ultrasonic probe 101 and the apparatus main body 100. However, the apparatus main body 100 is capable of connecting a plurality of ultrasonic probes. Which of the plurality of connected ultrasonic probes is used for ultrasonic scanning can be discretionarily selected by, for example, a software button on a touch panel to be described later.

The apparatus main body 100 is an apparatus which generates an ultrasonic image based on reflected wave signals received by the ultrasonic probe 101. The apparatus main body 100 includes an ultrasound transmission circuitry 110, an ultrasound reception circuitry 120, an internal storage circuitry 130, an image memory 140, an input interface 150, an output interface 160, a communication interface 170, and a processing circuitry 180.

The ultrasound transmission circuitry 110 is a processor which supplies a drive signal to the ultrasonic probe 101. The ultrasound transmission circuitry 110 is realized by, for example, a trigger generation circuitry, a delay circuitry, a pulsar circuitry, etc. The trigger generation circuitry repeatedly generates rate pulses for forming transmit ultrasonic waves at a predetermined rate frequency. The delay circuitry imparts, to each rate pulse generated by the trigger generation circuitry, a delay time for each plurality of piezoelectric vibrators necessary for determining transmission directivity by converging ultrasonic waves generated from the ultrasonic probe into a beam form. The pulsar circuitry applies drive signals (drive pulses) to the plurality of ultrasonic vibrators provided in the ultrasonic probe 101 at a timing based on the rate pulse. By varying the delay time to be imparted to each rate pulse by the delay circuitry, the transmission direction from the plurality of piezoelectric vibrator surfaces can be discretionarily adjusted.

Further, the ultrasound transmission circuitry 110 can discretionarily change an output intensity of ultrasonic waves by a drive signal. In the ultrasonic diagnostic apparatus, an influence of attenuation of ultrasonic waves in the living body P can be reduced by increasing the output intensity. The ultrasonic diagnostic apparatus can acquire a reflected wave signal having a large S/N ratio at the time of reception by reducing the influence of ultrasonic wave attenuation.

Generally, when ultrasonic waves propagate in the living body P, a vibration intensity (which is also referred to as acoustic power) of the ultrasonic waves, which corresponds to the output intensity, is attenuated. The attenuation of acoustic power is caused by absorption, scattering, reflection, etc. Also, a degree of decrease in acoustic power depends on a frequency of ultrasonic waves and a distance in a radiation direction of the ultrasonic waves. For example, by increasing the frequency of ultrasonic waves, the degree of attenuation increases. Further, the longer the distance in the radiation direction of the ultrasonic waves, the greater the degree of attenuation.

The ultrasound reception circuitry 120 is a processor which executes various processes on a reflected wave signal received by the ultrasonic probe 101, and generates a reception signal. The ultrasound reception circuitry 120 generates a reception signal for a reflected wave signal of ultrasonic waves acquired by the ultrasonic probe 101. Specifically, the ultrasound reception circuitry 120 is realized by, for example, a preamplifier, an A/D converter, a demodulator, a beamformer (adder), etc. The preamplifier amplifies a reflected wave signal received by the ultrasonic probe 101 for each channel and performs a gain correction process. The A/D converter converts the gain-corrected reflected wave signal into a digital signal. The demodulator demodulates the digital signal. The beamformer, for example, gives the demodulated digital signal a delay time necessary for determining reception directivity, and adds a plurality of digital signals that were given the delay times. By the addition process of the beamformer, a reception signal is generated in which a reflected component from a direction corresponding to the reception directivity is emphasized. The reception signal may also be referred to as an IQ signal. Further, the ultrasound reception circuitry 120 may store the reception signal (IQ signal) in the internal storage circuitry 130 to be described later, or may output the reception signal (IQ signal) to the external device 104 via the communication interface 170.

The internal storage circuitry 130 includes, for example, a storage medium which is readable by a processor, such as a magnetic or optical storage medium or a semiconductor memory. The internal storage circuitry 130 stores therein a program for realizing ultrasound transmission/reception, a program and various data related to transmit aperture synthesis control processing to be described later, etc. The programs and various data may be, for example, pre-stored in the internal storage circuitry 130. Further, the programs and various data may be, for example, stored and distributed in a non-volatile storage medium, and may be read from the non-volatile storage medium and installed into the internal storage circuitry 130. In addition, the internal storage circuitry 130 stores B-mode image data, contrast image data, image data related to blood flow images, etc. generated by the processing circuitry 180 according to an operation input through the input interface 150. The internal storage circuitry 130 can transfer the stored image data to the external device 104, etc. through the communication interface 170. The internal storage circuitry 130 may store the reception signal (IQ signal) generated by the ultrasound reception circuitry 120, or may transfer the reception signal (IQ signal) to the external device 104, etc. through the communication interface 170.

The internal storage circuitry 130 may be a drive device, etc. which reads and writes various information to and from a portable storage medium such as a CD drive, a DVD drive, and a flash memory. The internal storage circuitry 130 can write stored data into a portable storage medium, and store the data in the external device 104 via the portable storage medium.

The image memory 140 includes, for example, a storage medium which is readable by a processor, such as a magnetic or optical storage medium or a semiconductor memory. The image memory 140 stores image data corresponding to a plurality of frames immediately before a freeze operation which is input through the input interface 150. The image data stored in the image memory 140 is sequentially displayed (cine-displayed), for example.

The above-described internal storage circuitry 130 and image memory 140 may not necessarily be realized by independent storage devices. The internal storage circuitry 130 and the image memory 140 may be realized by a single storage device. Further, the internal storage circuitry 130 and the image memory 140 may each be realized by a plurality of storage devices.

The input interface 150 receives various instructions from an operator through the input device 102. The input device 102 is, for example, a mouse, a keyboard, a panel switch, a slider switch, a trackball, a rotary encoder, an operation panel, and a touch command screen (TCS). The input interface 150 is, for example, connected to the processing circuitry 180 via a bus, converts an operation instruction which is input by the operator into an electric signal, and outputs the electric signal to the processing circuitry 180. The input interface 150 is not limited to those connected to physical operation parts such as a mouse and a keyboard. For example, a circuitry which receives an electric signal corresponding to an operation instruction input from an external input device provided independently from the ultrasonic diagnostic apparatus 1, and outputs the electric signal to the processing circuitry 180, is also included in the examples of the input interface.

The output interface 160 is, for example, an interface for outputting an electric signal from the processing circuitry 180 to the output device 103. The output device 103 is a discretionary display, such as a liquid crystal display, an organic EL display, an LED display, a plasma display, or a CRT display. The output device 103 may be a touch panel type display which also serves as the input device 102. In addition to the display, the output device 103 may further include a speaker which outputs audio. The output interface 160 is, for example, connected to the processing circuitry 180 via a bus, and outputs the electric signal from the processing circuitry 180 to the output device 103.

The communication interface 170 is, for example, connected to the external device 104 via the network NW, and performs data communications with the external device 104.

The processing circuitry 180 is, for example, a processor which functions as the center of the ultrasonic diagnostic apparatus 1. The processing circuitry 180 executes a program stored in the internal storage circuitry 130, thereby realizing a function corresponding to the program. The processing circuitry 180 includes, for example, a B-mode processing function 181, a Doppler processing function 182, an image generation function 183, a timer function 184, a bubble speed detection function 185, a determination function 186, a beams to compound number determination function 187, a display control function 188, and a system control function 189.

The B-mode processing function 181 is a function of generating B-mode data, based on a reception signal received from the ultrasound reception circuitry 120. In the B-mode processing function 181, the processing circuitry 180 performs, for example, envelope detection processing, logarithmic compression processing, etc. on the reception signal received from the ultrasound reception circuitry 120 to generate data (B-mode data) in which a signal intensity is expressed by brightness. The generated B-mode data is stored in a RAW data memory (not shown) as B-mode RAW data on a two-dimensional ultrasonic scan line (raster).

Further, the processing circuitry 180 can execute a contrast echo method, e.g., contrast harmonic imaging (CHI), by the B-mode processing function 181. That is, the processing circuitry 180 can separate reflected wave data (a harmonic component or subharmonic component) of the living body P into which a contrast medium is injected and reflected wave data (a fundamental wave component) using a tissue in the living body P as a reflection source. Thereby, the processing circuitry 180 can extract the harmonic component or subharmonic component from the reflected wave data of the living body P to generate B-mode data for generating contrast image data.

The B-mode data for generating contrast image data is data in which a signal intensity of a reflected wave using the contrast medium as a reflection source is expressed by brightness. Further, the processing circuitry 180 can extract the fundamental wave component from the reflected wave data of the living body P, and generate B-mode data for generating tissue image data.

In an examination mode (contrast examination mode) using a contrast medium such as the above-described CHI, an examination is performed in a plurality of different time phases depending on the organ of interest. For example, if the organ of interest is the liver, the plurality of different time phases include, for example, a vascular phase and a post-vascular phase (Kupffer phase). The vascular phase is further divided into an arterial predominant phase and a portal predominant phase. In the vascular phase, blood flow imaging is performed in which a flow of contrast medium is regarded as a blood flow for examination. The Kupffer phase is a phase in which the contrast medium is taken up by Kupffer cells of the liver. In the Kupffer phase, Kupffer imaging is performed, which mainly examines the liver parenchyma.

When performing CHI, the processing circuitry 180 can extract a harmonic component by a method different from the above-described method using filter processing. In harmonic imaging, imaging methods called an amplitude modulation (AM) method, a phase modulation (PM) method, and an AMPM method, which is a combination of the AM method and the PM method, are performed.

With the AM method, PM method, and AMPM method, multiple ultrasound transmissions with different amplitudes and phases are performed to the same scan line. Thereby, the ultrasound reception circuitry 120 generates a plurality of reflected wave data at each scan line, and outputs the generated reflected wave data. The processing circuitry 180 extracts a harmonic component by performing addition/subtraction processing on a plurality of reflected wave data of each scan line according to a modulation method by the B-mode processing function 181. Then, the processing circuitry 180 performs envelope detection processing, etc. on the reflected wave data of the harmonic component to generate B-mode data.

The Doppler processing function 182 is a function of analyzing a frequency of a reception signal received from the ultrasound reception circuitry 120 so as to generate data (Doppler information) in which motion information based on the Doppler effect of a moving object in a region of interest (ROI) set in a scan area is extracted. The generated Doppler information is stored in a RAW data memory (not shown) as Doppler RAW data (also referred to as Doppler data) on a two-dimensional ultrasonic scan line.

Specifically, the processing circuitry 180 estimates, for example, an average velocity, an average dispersion value, an average power value, etc. at each of a plurality of sample points as motion information of a moving object, and generates Doppler data showing the estimated motion information, using the Doppler processing function 182. The moving object is, for example, a blood flow, a tissue such as a cardiac wall, and a contrast medium. The processing circuitry 180 according to the first embodiment estimates, using the Doppler processing function 182, an average blood flow velocity, a blood flow velocity dispersion value, a power value of a blood flow signal, etc. at each of a plurality of sample points as blood flow motion information (blood flow information), and generates Doppler data showing the estimated blood flow information.

The image generation function 183 is a function of generating B-mode image data based on the data generated by the B-mode processing function 181. For example, in the image generation function 183, the processing circuitry 180 converts (scan-converts) a scan line signal string of ultrasonic scanning into a scan line signal string of a video format typified by a television, etc., and generates image data for display (display image data). Specifically, the processing circuitry 180 executes RAW-pixel conversion, e.g., coordinate conversion according to a scanning form of ultrasonic waves by the ultrasonic probe 101, for B-mode RAW data stored in the RAW data memory, thereby generating two-dimensional B-mode image data (also referred to as ultrasonic image data) composed of pixels. In other words, the processing circuitry 180 generates a plurality of ultrasonic images (medical images) respectively corresponding to a plurality of consecutive frames through transmission and reception of ultrasonic waves by the image generation function 183.

In addition, the processing circuitry 180 executes, for example, RAW-pixel conversion on the Doppler RAW data stored in the RAW data memory, thereby generating Doppler image data in which the blood flow information is visualized. The Doppler image data is average velocity image data, dispersion image data, power image data, or image data obtained by a combination thereof. As the Doppler image data, the processing circuitry 180 generates color Doppler image data in which blood flow information is displayed in color and Doppler image data in which one piece of blood flow information is displayed in a wavy shape on a gray scale. The color Doppler image data is generated when the above-described blood flow image mode is executed.

The timer function 184 is a function of measuring a time. For example, in the timer function 184, the processing circuitry 180 receives a time measurement start instruction from a user. Then, the processing circuitry 180 measures the time starting from the time when the start instruction is received.

The bubble speed detection function 185 is a function of tracking each microbubble used as a contrast medium in the contrast echo method and performing autocorrelation processing for each frame to detect a moving speed (speed of the bubble) of the contrast medium. For example, in the bubble speed detection function 185, the processing circuitry 180 detects the moving speed of the contrast medium from a time difference between adjacent frames and a distance of a bubble being tracked.

The determination function 186 is a function of determining whether or not to increase the number of beams to be compounded in transmit aperture synthesis. For example, in the determination function 186, the processing circuitry 180 determines whether or not to increase the number of beams to be compounded based on information regarding the examination mode. The information regarding the examination mode includes, for example, speed information regarding a speed of a bubble contained in a contrast medium, an elapsed time based on a time when administration of the contrast medium to a subject is started, etc. Specifically, the processing circuitry 180 determines that the number of beams to be compounded is to be increased when the bubble speed is equal to or less than a predetermined speed (threshold value). Further, the processing circuitry 180 determines that the number of beams to be compounded is to be increased when the elapsed time by the timer function 184 is a predetermined time (e.g., 10 minutes) or more.

The beams to compound number determination function 187 is a function of determining the number of beams to be compounded related to transmit aperture synthesis after it is determined that the number of beams to be compounded is to be increased. In the beams to compound number determination function 187, the processing circuitry 180 increases the number of beams to be compounded when a predetermined condition is satisfied. Specifically, when a current frame rate is equal to or higher than a predetermined frame rate, when a signal intensity of contrast image data in a current number of compounded beams is equal to or higher than a signal intensity of contrast image data in an immediately preceding number of compounded beams, and when the signal intensity is not saturated, the processing circuitry 180 further increases the number of beams to be compounded. Further, when at least one of the above-described predetermined conditions is not satisfied, the processing circuitry 180 determines the immediately preceding number of compounded beams immediately before the current number of compounded beams. For example, the processing circuitry 180 determines the number of beams to be compounded to be “4” when the number of compounded beams is increased from “4” to “5” immediately prior.

The display control function 188 is a function of displaying an image based on various ultrasonic image data generated by the image generation function 183 on a display as the output device 103. Specifically, for example, the processing circuitry 180 controls, by the display control function 188, displays on a display of an image based on B-mode image data, contrast image data, or image data including both of them, generated by the image generation function 183.

More specifically, the processing circuitry 180 converts (scan converts), by the display control function 188, for example, a scan line signal string of ultrasonic scanning into a scan line signal string of a video format typified by a television, etc., and generates display image data. In addition, the processing circuitry 180 may perform various processes, such as dynamic range, brightness, contrast, and y curve corrections, and RGB conversion, on the display image data. The processing circuitry 180 may add supplementary information, such as textual information of various parameters, a scale, or a body mark, to the display image data. In addition, the processing circuitry 180 may generate a user interface (GUI: Graphical User Interface) for an operator to input various instructions through the input device, and display the GUI on a display.

Further, by the display control function 188, the processing circuitry 180 may display a plurality of contrast image data related to transmit aperture synthesis on one screen. Specifically, the processing circuitry 180 may simultaneously display contrast image data in which transmit aperture synthesis is performed and contrast image data in which transmit aperture synthesis is not performed. Further, the processing circuitry 180 may simultaneously display a plurality of contrast image data different in number of compounded beams.

The system control function 189 is a function of controlling operations of the entire ultrasonic diagnostic apparatus 1 in an integrated manner. For example, in the system control function 189, the processing circuitry 180 controls the ultrasound transmission circuitry 110 and the ultrasound reception circuitry 120 to execute scanning for transmit aperture synthesis during execution of an examination mode (contrast examination mode) using a contrast medium.

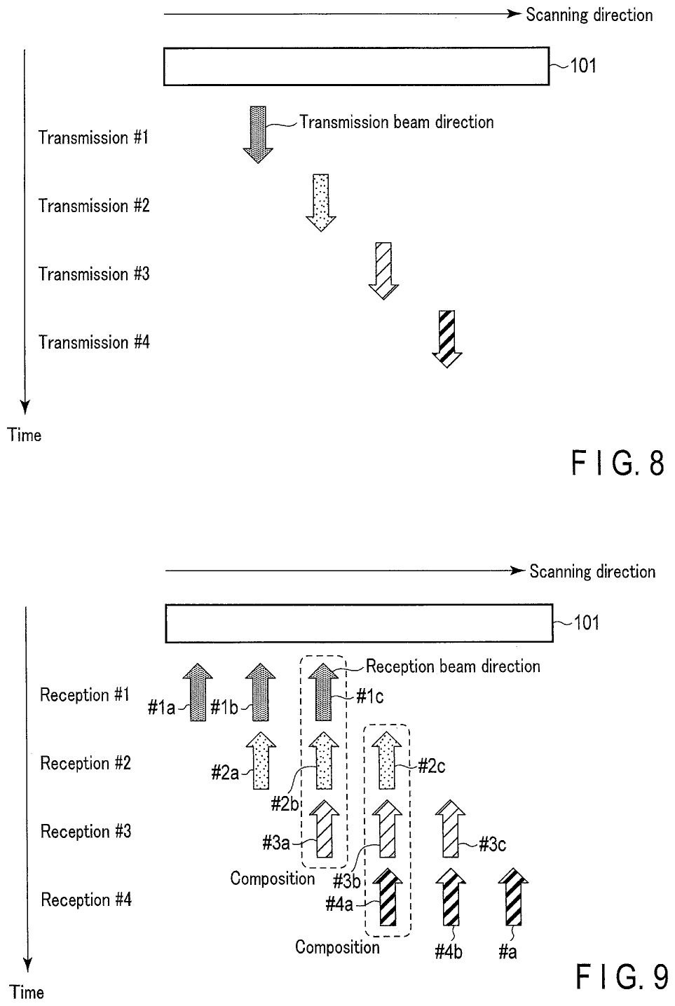

In the above, the basic configuration of the ultrasonic diagnostic apparatus 1 according to the first embodiment has been described. Next, transmit aperture synthesis which can be executed by the ultrasonic diagnostic apparatus 1 according to the first embodiment will be described with reference to FIGS. 8 and 9.

FIG. 8 is a diagram for explaining operations of transmit control through transmit aperture synthesis. The example of FIG. 8 shows a case where the ultrasonic probe 101 transmits ultrasonic waves four times in order of transmission #1, transmission #2, transmission #3, and transmission #4, by shifting a transmit focal position.

FIG. 9 is a diagram for explaining operations of receive control through transmit aperture synthesis. The example of FIG. 9 shows a case where the ultrasonic probe 101 receives reflected wave signals in response to each of the ultrasound transmissions of transmission #1 to transmission #4 of FIG. 8, and generates three reception signals each having different directivity. In the example of FIG. 9, the ultrasonic probe 101 receives reflected wave signals in the order of reception #1, reception #2, reception #3, and reception #4, respectively corresponding to ultrasound transmissions of transmissions #1 to #4.

Specifically, at reception #1, the ultrasonic diagnostic apparatus 1 generates reception signals #1a, #1b, and #1c, in response to ultrasound transmission of transmission #1. At reception #2, the ultrasonic diagnostic apparatus 1 generates reception signals #2a, #2b, and #2c, in response to ultrasound transmission of transmission #2. Similarly, at reception #3, the ultrasonic diagnostic apparatus 1 generates reception signals #3a, #3b, and #3c, in response to ultrasound transmission of transmission #3. At reception #4, the ultrasonic diagnostic apparatus 1 generates reception signals #4a, #4b, and #4c, in response to ultrasound transmission of transmission #4. Hereinafter, the number of a plurality of reception signals in response to one transmission will be referred to as the number of simultaneous receptions. For example, in the case of FIG. 9, the number of simultaneous receptions is “3”.

Then, the ultrasonic diagnostic apparatus 1 compounds reception signals in the same channel obtained through different transmissions. For example, as shown in FIG. 9, the ultrasonic diagnostic apparatus 1 compounds the reception signals #1c, #2b, and #3a at different transmit apertures and in the same scan line. Further, for example, the ultrasonic diagnostic apparatus 1 compounds the reception signals #2c, #3b, and #4a at different transmit apertures and in the same scan line. The number of compounded beams described above corresponds to the number of compounded reception signals of the same channel obtained through different transmissions.

The number of compounded beams is not limited to three as illustrated in FIG. 9, and may be two, or four or more. In this case, the number of simultaneous receptions increases or decreases according to the number of compounded beams. Further, the number of simultaneous receptions and the number of compounded beams do not have to match. For example, when the number of simultaneous receptions is “5”, the number of compounded beams may be “3”. To summarize the above, the ultrasonic diagnostic apparatus 1 may change the number of simultaneous receptions in scanning for transmit aperture synthesis according to the number of compounded beams, or may set the number of simultaneous receptions in scanning for transmit aperture synthesis to be constant regardless of the number of compounded beams.

In the above-described transmit aperture synthesis, an example has been described in which the transmission position and the reception position of the ultrasonic probe 101 are shifted for scanning, but the present invention is not limited thereto. For example, in transmit aperture synthesis, at least one of the transmission position and the reception position may be fixed. For example, when both the transmission position and the reception position are fixed, the ultrasonic diagnostic apparatus 1 may consider only the number of compounded beams.

In the above, the transmit aperture synthesis which can be executed by the ultrasonic diagnostic apparatus 1 according to the first embodiment has been described. Next, operations of the ultrasonic diagnostic apparatus 1 according to the first embodiment will be described.

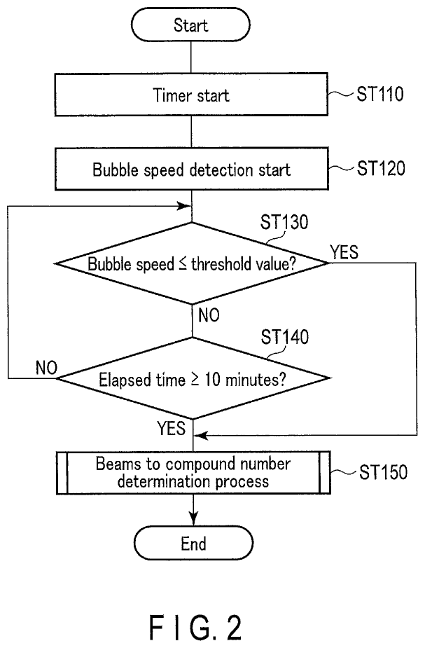

FIG. 2 is a flowchart for explaining operations of the processing circuitry which executes transmit aperture synthesis control processing according to the first embodiment. The transmit aperture synthesis control processing in the first embodiment performs optimum control when executing transmit aperture synthesis during execution of an examination mode (contrast examination mode) using a contrast medium. The transmit aperture synthesis control processing shown in FIG. 2 is, for example, started when a user executes the contrast examination mode. In the following, as a specific example, a contrast examination of the liver will be performed.

(Step ST110)

When the transmit aperture synthesis control processing is started, the processing circuitry 180 executes the timer function 184. When the timer function 184 is executed, the processing circuitry 180 receives a time measurement start instruction from the user. The processing circuitry 180 measures the time starting from the time when the start instruction is received. At this time, the user gives the time measurement start instruction approximately at the same time as administration of the contrast medium to a subject. Thus, the measured time corresponds to an elapsed time from the start of administration of the contrast medium to the subject.

(Step ST120)

Approximately at the same time as starting the time measurement, the processing circuitry 180 executes the bubble speed detection function 185. When the bubble speed detection function 185 is executed, the processing circuitry 180 tracks the microbubbles used as the contrast medium for each frame, and detects a bubble speed. Note that step ST120 may be performed before step ST110, or may be performed approximately at the same time as step ST110.

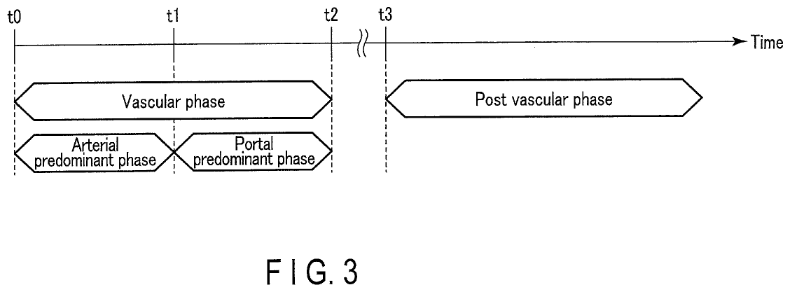

FIG. 3 is a diagram for explaining a flow of an examination in the contrast examination mode in the first embodiment. FIG. 3 shows a plurality of time phases according to dynamics of the contrast medium for the contrast examination of the liver. Time t0 is a start time of administration of the contrast medium to the subject. A time phase between time t0 and time t1 is called an arterial predominant phase, and a time phase between time t1 and time t2 is called a portal predominant phase. As described above, the time phase between the time to and the time t2 is also called a vascular phase. A time phase at and after time t3 is called a post-vascular phase (Kupffer phase). Times from time t1 to time t3 are, for example, 30 seconds, 180 seconds, and 600 seconds (10 minutes), respectively.

In the arterial predominant phase, the user observes an initial image of contrast medium administration (e.g., blood flow image and perfusion image) for a main lesion (hepatic nodule). Next, in the portal predominant phase, the user observes a reperfusion image for the hepatic nodule, if necessary. At this time, the user can observe the reperfusion image by performing a flash scan using a Flash Replenishment method. Note that the Kupffer phase will be described later.

(Step ST130)

After the observation in the vascular phase is finished, the processing circuitry 180 executes the determination function 186. When the determination function 186 is executed, the processing circuitry 180 determines the bubble speed. Specifically, the processing circuitry 180 determines whether or not the bubble speed is equal to or less than a threshold value. If it is determined that the bubble speed is equal to or less than the threshold value, the process proceeds to step ST140, and if it is determined that the bubble speed is not equal to or less than the threshold value, the process proceeds to step ST150. The threshold value here may be discretionarily determined.

At this time, it is assumed that the user has started scanning using the probe without waiting for an elapsed time of 10 minutes. As described above, the Kupffer phase is assumed to be 10 minutes after administration of the contrast medium. However, since it is possible to examine the liver parenchyma if there is no movement of bubbles due to blood flow, it is thought that the user can perform observation in the Kupffer phase without waiting for the elapsed time of 10 minutes in some cases.

(Step ST140)

After determining that the bubble speed is not equal to or less than the threshold value, the processing circuitry 180 determines whether or not the elapsed time is 10 minutes or more using the determination function 186. If it is determined that the elapsed time is not 10 minutes or more, the process returns to step ST130, and if it is determined that the elapsed time is 10 minutes or more, the process proceeds to step ST150.

(Step ST150)

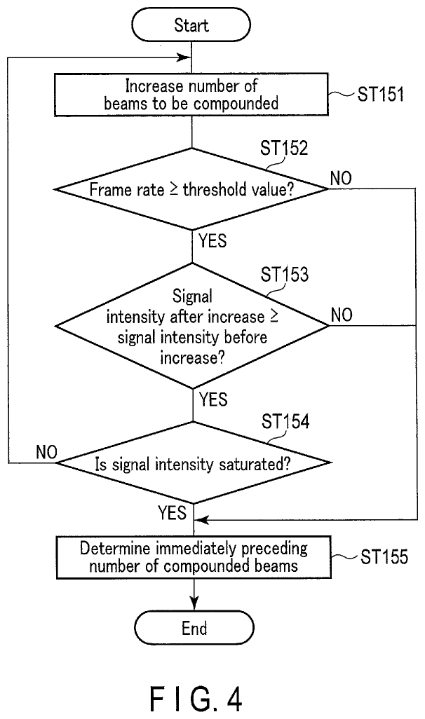

After determining that the bubble speed is equal to or less than the threshold value in step ST130, or after determining that the elapsed time is 10 minutes or more in step ST140, the processing circuitry 180 executes the beams to compound number determination function 187. When the beams to compound number determination function 187 is executed, the processing circuitry 180 performs a beams to compound number determination process including a process of increasing the number of beams to be compounded. Hereinafter, a specific example of the beams to compound number determination process will be described with reference to the flowchart of FIG. 4.

FIG. 4 is a flowchart illustrating the beams to compound number determination process of the flowchart of FIG. 2. The flowchart of FIG. 4 describes the details of the process of step ST150 in FIG. 2.

(Step ST151)

After determining that the bubble speed is equal to or less than the threshold value in step ST130, or after determining that the elapsed time is 10 minutes or more in step ST140, the processing circuitry 180 increases the number of beams to be compounded using the beams to compound number determination function 187. Specifically, the processing circuitry 180 adds “1” to an immediately preceding number of compounded beams. For example, if the immediately preceding number of compounded beams is “3”, the processing circuitry 180 sets the number of beams to be compounded to “4”. If the immediately preceding number of compounded beams is “1”, it means that transmit aperture synthesis is not performed.

(Step ST152)

After increasing the number of beams to be compounded, the processing circuitry 180 determines whether or not a frame rate is equal to or higher than a threshold value. If it is determined that the frame rate is equal to or higher than the threshold value, the process proceeds to step ST153, and if it is determined that the frame rate is not equal to or higher than the threshold value, the process proceeds to step ST155. This threshold value is a frame rate when observing the Kupffer phase, and is, for example, about 10 fps.

(Step ST153)

After determining that the frame rate is equal to or higher than the threshold value, the processing circuitry 180 determines whether or not a signal intensity of contrast image data is improved. Specifically, the processing circuitry 180 determines whether or not a signal intensity after increasing the number of beams to be compounded (a signal intensity after the increase) is equal to or higher than a signal intensity before increasing the number of beams to be compounded (a signal intensity before the increase). If it is determined that the signal intensity after the increase is equal to or higher than the signal intensity before the increase, the process proceeds to step ST154, and if it is determined that the signal intensity after the increase is not equal to or higher than the signal intensity before the increase, the process proceeds to step ST155.

(Step ST154)

After determining that the signal intensity after the increase is equal to or higher than the signal intensity before the increase, the processing circuitry 180 determines whether or not the signal intensity of the contrast image data is saturated. If it is determined that the signal intensity is not saturated, the process returns to step ST151, and if it is determined that the signal intensity is saturated, the process proceeds to step ST155.

(Step ST155)

After determining in step ST152 that the frame rate is not equal to or higher than the threshold value, after determining in step ST153 that the signal intensity after the increase is equal to or higher than the signal intensity before the increase, or after determining in step ST154 that the signal intensity after the increase is not equal to or higher than the signal intensity before the increase, the processing circuitry 180 determines the immediately preceding number of compounded beams as the number of beams to be compounded. For example, the processing circuitry 180 determines that the number of beams to be compounded is “4” when the process of step ST155 is performed in a state where the number of compounded beams is “5”. After step ST155, the beams to compound number determination process and the transmit aperture synthesis control processing of the flowchart of FIG. 2 are ended.

To summarize the processes of the flowchart of FIG. 4, the processing circuitry 180 repeats the process of increasing the number of beams to be compounded in step ST151 as long as the predetermined conditions from step ST152 to step ST154 are satisfied.

The above-described steps ST152 to ST154 may be discretionarily replaced. For example, when the process of step ST152 or the process of step ST153 is replaced with the position of step ST154, the process returns to step ST151 in the determination of “YES” of step ST154. Further, for example, when the process of step ST154 is replaced with the position of step ST152, the process proceeds to step ST153 at the determination of “NO” of step ST152. Similarly, when the process of step ST154 is replaced with the position of step ST153, the process proceeds to step ST154 in the determination of “NO” of step ST153.

In the flowcharts of FIGS. 2 and 4, the determinations in step ST130 and step ST140 are the same as the determination as to whether or not to make a transition to step ST151. That is, the determinations in step ST130 and step ST140 can be regarded as determining whether or not to perform the process of increasing the number of beams to be compounded in step ST151.

(Display Example of Contrast Image Data)

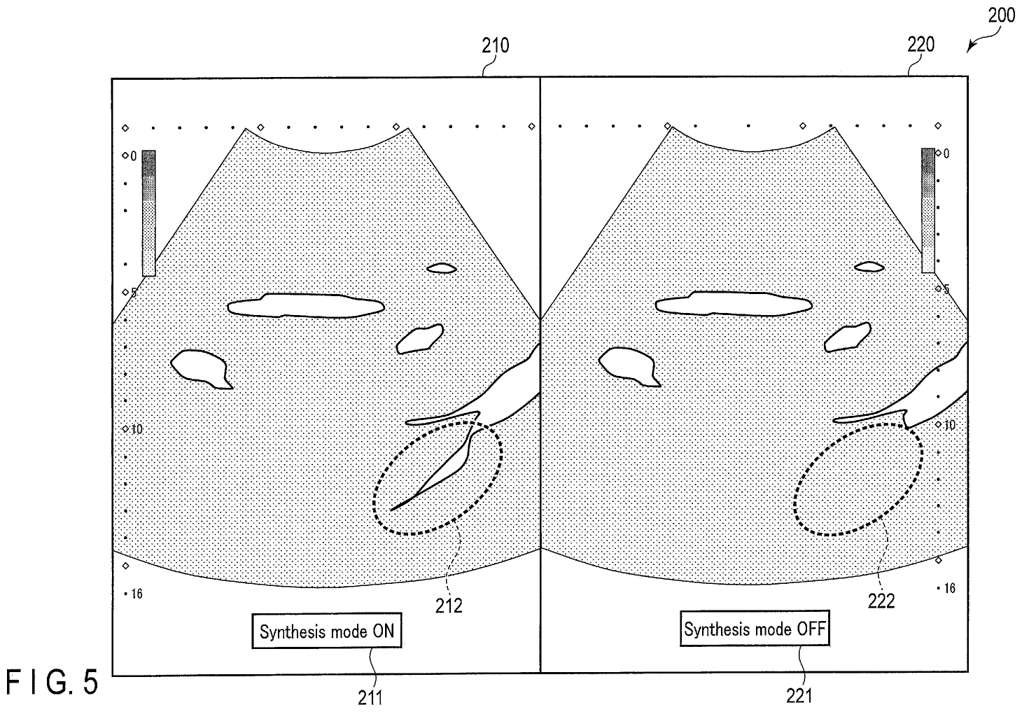

FIG. 5 is a diagram showing a first display example of contrast image data in the first embodiment. In display image data 200 of FIG. 5, first contrast image data 210 and second contrast image data 220 are displayed side by side at the same time. The first contrast image data 210 is contrast image data in which transmit aperture synthesis is performed, and has an icon 211 “synthesis mode ON”. The second contrast image data 220 is contrast image data in which transmit aperture synthesis is not performed, and has an icon 221 “synthesis mode OFF”.

At this time, the user can easily confirm a difference in image depending on the presence or absence of transmit aperture synthesis by viewing the display image data 200. For example, in the first contrast image data 210, the presence of a deep structure can be discerned in a region 212, but in the second contrast image data 220, a deep structure is buried in white noise in a region 222 at the same position as that of the region 212 and visibility is poor. This is because, by performing the transmit aperture synthesis, an S/N ratio is expected to improve, so a noise level of the deep portion is reduced, and the visibility of the structure that was difficult to see due to noise is improved. In addition, the user can select an image suitable for a medical examination.

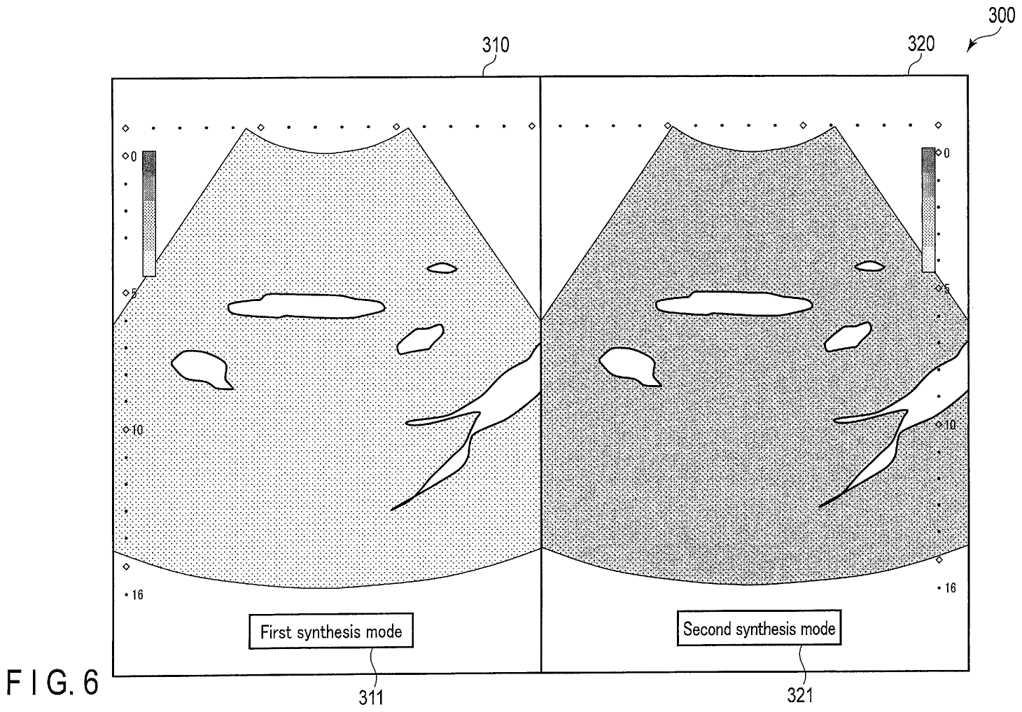

FIG. 6 is a diagram showing a second display example of contrast image data in the first embodiment. In display image data 300 of FIG. 6, first contrast image data 310 and second contrast image data 320 are displayed side by side at the same time. The first contrast image data 310 is contrast image data in which transmit aperture synthesis is performed with a first number of beams to be compounded, and has an icon 311 “First synthesis mode”. The second contrast image data 320 is contrast image data in which transmit aperture synthesis is performed with a second number of beams to be compounded larger than the first number of beams to be compounded, and has an icon 321 “Second synthesis mode”.

At this time, the user can easily confirm a difference in image due to the difference in number of compounded beams by viewing the display image data 300. For example, when comparing the first contrast image data 310 and the second contrast image data 320, a contrast of the second contrast image data 320 is higher than that of the first contrast image data 310. This allows the user to select an image suitable for a medical examination.

(Timer Reset Process)

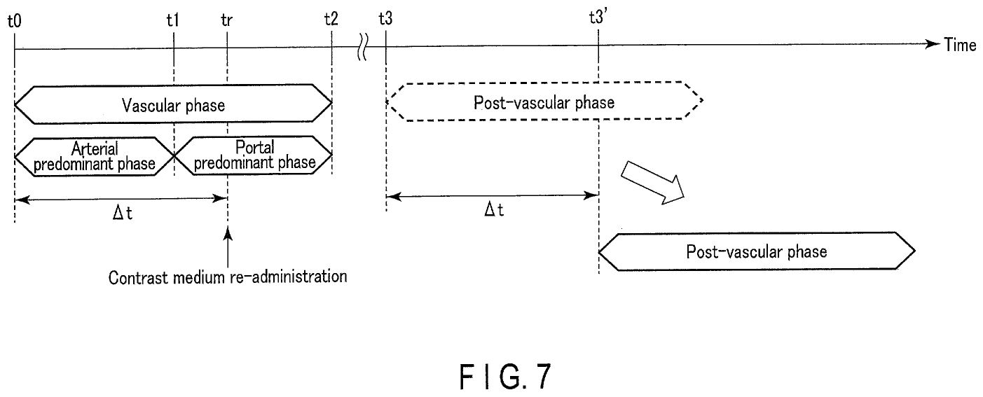

FIG. 7 is a diagram for explaining the timer reset process in the first embodiment. For example, in a case of re-administering a contrast medium because a specified number of times or more of flash scans are performed for any reason during a contrast examination, it is necessary to redo the time measurement in accordance with the re-administration of the contrast medium. As shown in FIG. 7, for example, in a case of performing re-administration of a contrast medium at time tr between time t1 and time t2, the user gives a time measurement start instruction again. At this time, the processing circuitry 180 measures the time starting from time tr. Alternatively, the processing circuitry 180 may newly set time t3′ obtained by adding a time difference Δt from time t0 to time tr to time t3. Summarizing the above, the timer reset process has cases of performing remeasurement and offsetting the measurement time. In the above manner, by performing the timer reset process, the processing circuitry 180 can appropriately determine a start time of a post-vascular phase (Kupffer phase) even in a case of contrast medium re-administration.

As described above, the ultrasonic diagnostic apparatus according to the first embodiment can execute scanning for transmit aperture synthesis during execution of an examination mode using a contrast medium, and determine the number of beams to be compounded related to the transmit aperture synthesis based on information on the examination mode.

Therefore, since the ultrasonic diagnostic apparatus according to the first embodiment can appropriately determine the number of beams to be compounded related to the transmit aperture synthesis during execution of the contrast examination mode, generation of a contrast image having excellent spatial resolution and contrast resolution can be expected. Thereby, the user can, for example, easily discern a shape of a tumor in the contrast image, and thus examination accuracy in the contrast examination can be improved.

### Other Embodiments

In the above-described first embodiment, a process of determining the number of beams to be compounded related to transmit aperture synthesis in a post-vascular phase (Kupffer phase) (beams to compound number determination process) is performed, but the present invention is not limited thereto. For example, as long as a predetermined condition is satisfied, a beams to compound number determination process may be performed in a vascular phase. The predetermined condition is, for example, to suppress an influence of bubble speed in beam composition. Specifically, the processing circuitry 180 executes the process of step ST150 after step ST120 in FIG. 2. Thereby, the processing circuitry 180 can execute a beams to compound number determination process in the vascular phase.

According to at least one of the above-described embodiments, examination accuracy in a contrast examination can be improved.

The term “processor” used in the descriptions of the embodiments means, for example, circuitry such as a central processing unit (CPU), a graphics processing unit (GPU), an application specific integrated circuit (ASIC), a programmable logic device (e.g., a simple programmable logic device (SPLD)), a complex programmable logic device (CPLD), and a field programmable gate array (FPGA). The processor reads and executes programs stored in the storage circuitry to realize respective functions. The programs may be incorporated directly into circuitry of the processor, instead of storing them in the storage circuitry. In this case, the processor reads the programs incorporated into the circuitry and executes them to realize the functions. The processors of the above-described embodiments are not limited to single-circuit processors. A plurality of independent circuits may be combined and integrated as one processor to realize the functions. Furthermore, a plurality of constituent elements in the above-described embodiments may be integrated into one processor to realize the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasonic diagnostic apparatus comprising processing circuitry configured to:
execute scanning for transmit aperture synthesis during execution of an examination mode using a contrast medium; and
determine a number of beams to be compounded related to the transmit aperture synthesis based on information on the examination mode.

2. The ultrasonic diagnostic apparatus according to claim 1, wherein the processing circuitry is further configured to:
determine whether or not to increase the number of beams to be compounded based on the information on the examination mode; and
increase the number of beams to be compounded when determining that the number of beams to be compounded is to be increased.

3. The ultrasonic diagnostic apparatus according to claim 2, wherein the information on the examination mode includes speed information on a speed of a bubble contained in the contrast medium, and wherein the processing circuitry is further configured to:
compare the speed information and a predetermined speed, and
increase the number of beams to be compounded when the speed information is equal to or less than the predetermined speed.

4. The ultrasonic diagnostic apparatus according to claim 2, wherein the information on the examination mode includes an elapsed time based on a time when the contrast medium is started to be administered to a subject, and wherein the processing circuitry is further configured to:
compare the elapsed time and a predetermined time; and
increase the number of beams to be compounded when the elapsed time is equal to or more than the predetermined time.

5. The ultrasonic diagnostic apparatus according to claim 4, wherein the predetermined time is 10 minutes.

6. The ultrasonic diagnostic apparatus according to claim 4, wherein the processing circuitry is further configured to, when the elapsed time is reset, compare an elapsed time based on a time when the elapsed time is reset and the predetermined time.

7. The ultrasonic diagnostic apparatus according to claim 2, wherein the processing circuitry is further configured to increase the number of beams to be compounded based on a frame rate to display contrast image data and a signal intensity of the contrast image data.

8. The ultrasonic diagnostic apparatus according to claim 7, wherein the processing circuitry is further configured to determine an immediately preceding number of compounded beams immediately before a current number of compounded beams when the frame rate is less than a predetermined frame rate.

9. The ultrasonic diagnostic apparatus according to claim 7, wherein the processing circuitry is further configured to, when a signal intensity of contrast image data in a current number of compounded beams is less than a signal intensity of contrast image data in an immediately preceding number of compounded beams immediately before the current number of compounded beams, determine the immediately preceding number of compounded beams.

10. The ultrasonic diagnostic apparatus according to claim 7, wherein the processing circuitry is further configured to, when a signal intensity of contrast image data in a current number of compounded beams is saturated, determine an immediately preceding number of compounded beams immediately before the current number of compounded beams.

11. The ultrasonic diagnostic apparatus according to claim 1, wherein the processing circuitry is further configured to display a plurality of contrast image data related to the transmit aperture synthesis.

12. The ultrasonic diagnostic apparatus according to claim 11, wherein the processing circuitry is further configured to simultaneously display contrast image data in which the transmit aperture synthesis is performed and contrast image data in which the transmit aperture synthesis is not performed.

13. The ultrasonic diagnostic apparatus according to claim 11, wherein the processing circuitry is further configured to simultaneously display contrast image data in a first number of beams to be compounded and contrast image data in a second number of beams to be compounded larger than the first number of beams to be compounded.

14. The ultrasonic diagnostic apparatus according to claim 1, wherein the processing circuitry is further configured to change a number of simultaneous receptions in scanning for the transmit aperture synthesis according to the number of beams to be compounded.

15. The ultrasonic diagnostic apparatus according to claim 1, wherein the processing circuitry is further configured to set a number of simultaneous receptions in scanning for the transmit aperture synthesis to be constant regardless of the number of beams to be compounded.

16. A method, comprising:
executing scanning for transmit aperture synthesis during execution of an examination mode using a contrast medium; and
determining a number of beams to be compounded related to the transmit aperture synthesis based on information on the examination mode.
